(19) United States
(12) Patent Application Publication
(10) Pub. No.: US 2016/0166832 A1
(43) Pub. Date: Jun. 16, 2016

(54) Efferent Contraction Training Device And Training Method Using The Same

(71) Applicant: YONSEI UNIVERSITY WONJU INDUSTRY ACADEMIC COOPERATION FOUNDATION, Gangwon-do (KR)

(72) Inventors: Young-ho Kim, Wonju-si (KR); Jong-sang Son, Changwon-si (KR); Dongyeop Lee, Bucheon-si (KR); Je-seong Ryu, Wonju-si (KR)

(73) Assignee: Yonsei University Wonju Industry-Academic Cooperation Foundation, Wonju-si, KW (KR)

(21) Appl. No.: 14/902,686

(22) PCT Filed: Apr. 14, 2014
(86) PCT No.: PCT/KR2014/003178

§ 371 Date: Jan. 4, 2016

(30) Foreign Application Priority Data
Jul. 10, 2013 (KR) 10-2013-0081061

(51) Int. Cl. A61N 1/36

(52) U.S. Cl. CPC A61N 1/36003; A61N 1/36014

(57) ABSTRACT

An efferent contraction training device may include a fixing unit, an electrical stimulation pad and a driving unit. The fixing unit may be configured to fix an articulation of a patient. The fixing unit may include a first member and a second member connected to the first member. The second member of the fixing unit may have an angle with respect to the first member changed in accordance with movements of the articulation of the patient. The electrical stimulation pad may apply an electrical stimulation to a muscle of the articulation to provide the articulation with an efferent contraction exercise. The driving unit may drive the electrical stimulation pad. Thus, the efferent contraction training device may be effectively used for rehabilitating the patient with the paralytic musculature.

### CROSS-RELATED APPLICATION

This application claims priority under 35 USC §119 to Korean Patent Application No. 2013-0081061, filed on Jul. 10, 2013 in the Korean Intellectual Property Office (KIPO), the contents of which are herein incorporated by reference in their entirety.

### BACKGROUND

1. Field

Example embodiments relate to an efferent contraction training device. More particularly, example embodiments relate to an efferent contraction training device used for rehabilitating a patient with a paralytic musculature, and a training method using the efferent contraction training device.

2. Description of the Related Art

A brain-disabled patient may be numerous. Most of the brain-disabled patients may have a neurodegenerative disease such as a muscular paralysis. A training method using an electrical stimulation may be used for rehabilitating the patients with the paralytic musculature.

However, because the rehabilitation using the electrical stimulation may use simple muscle stimulation, the rehabilitation using the muscle stimulation may not be effectively used for the patient with the paralytic musculature.

### SUMMARY

Example embodiments provide an efferent contraction training device used for a patient with a paralytic musculature.

Example embodiments also provide a training method using the above-mentioned efferent contraction training device.

According to some example embodiments, there may be provided an efferent contraction training device. The efferent contraction training device may include a fixing unit, an electrical stimulation pad and a driving unit. The fixing unit may be configured to fix an articulation of a patient. The fixing unit may include a first member and a second member connected to the first member. The second member of the fixing unit may have an angle with respect to the first member changed in accordance with movements of the articulation of the patient. The electrical stimulation pad may apply an electrical stimulation to a muscle of the articulation to provide the articulation with an efferent contraction exercise. The driving unit may drive the electrical stimulation pad.

In example embodiments, the driving unit may include a control display panel. The driving unit may be connected to the fixing unit. The driving unit may measure torques generated by changing the angle between the first member and the second member of the fixing unit. The control display panel may display the torques.

In example embodiments, the driving unit may be connected to the fixing unit to rotate the first member and the second member of the fixing unit.

In example embodiments, the first member of the fixing unit may include a first frame, a first cushion pad configured to support the articulation of the patient, and a first fixing band configured to fix the articulation of the patient on the first cushion pad. The second member of the fixing unit may include a second frame, a second cushion pad configured to support the articulation of the patient, and a second fixing band configured to fix the articulation of the patient on the second cushion pad.

In example embodiments, the efferent contraction device may further include a table, a first supporting rod, a second supporting rod and a connecting rod. The table may be configured to support the fixing unit and the driving unit. The first supporting rod may be arranged under the table to support the table. The second supporting rod may be arranged under the first supporting rod to support the first supporting rod. The connecting rod may be connected between the first connecting rod and the second connecting rod to adjust lengths of the first connecting rod and the second connecting rod.

In example embodiments, the efferent contraction device may further include an angle-adjusting unit arranged between the table and the first supporting rod to adjust an angle between the table and the first supporting rod.

In example embodiments, the efferent contraction exercise may include a uniform motion.

In example embodiments, the electrical stimulation pad may apply a voltage to the articulation of the patient. A wave form of the voltage may include a heteromorphic square wave having a frequency of about 20 Hz and a dwell time of about 200 μs between about +20V and about −20V.

According to some example embodiments, there may be provided an efferent contraction training method. In the efferent contraction training method, a training program suitable for a patient may be set. An efferent contraction training using an electrical stimulation may be performed. The efferent contraction training may be repeated in accordance with the training program. Setting the training program may include determining a voltage waveform of the electrical stimulation, voltage intensity, an exercise time, an exercise interval and an exercise period. Performing the efferent contraction training may include applying an electrical stimulation to an articulation of the patient to provide the articulation of the patient with an efferent contraction exercise. Repeating the efferent contraction training may include continuing the efferent contraction training for the exercise time, and repeating the efferent contraction training the exercise numbers by the exercise interval.

In example embodiments, performing the efferent contraction training may include performing a uniform motion having an angular speed of about 10°/s.

In example embodiments, applying the electrical stimulation may include applying a heteromorphic square wave having a frequency of about 20 Hz and a dwell time of about 200 μs between about +20V and about −20V.

In example embodiments, the exercise time may be about 30 minutes. The exercise interval may be twice a week. The exercise period may be about 12 weeks.

According to example embodiments, the efferent contraction training device may provide the articulation of the patient with the efferent contraction exercise having a slow speed. The efferent contraction exercise having the slow speed may provide the articulation with a muscular tension stronger than a muscular tension generated by other exercises. Thus, the efferent contraction training device may be effectively used for rehabilitating the patient with the paralytic musculature.

Further, the efferent contraction training device may include the table having variable length and angle so that the efferent contraction training device may be adjusted to a body type of the patient.

Furthermore, the driving unit of the efferent contraction training device may measure the torques generated by the exercise of the patient and apply a proper load for setting an exercise intensity of the patient.

Moreover, the electrical stimulation pad of the efferent contraction training device may allow the articulation of the patient for the efferent contraction exercise having the slow uniform motion.

### BRIEF DESCRIPTION OF THE DRAWINGS

Example embodiments will be more clearly understood from the following detailed description taken in conjunction with the accompanying drawings. FIGS. 1 to 7 represent non-limiting, example embodiments as described herein.

FIG. 1 is a perspective view illustrating an efferent contraction training device in accordance with example embodiments;

FIG. 2 is a plan view illustrating the efferent contraction training device in FIG. 1;

FIG. 3 is a side view illustrating the efferent contraction training device in FIG. 1;

FIGS. 4A and 4B are perspective views illustrating the efferent contraction training device in FIG. 1 applied to an arm of a patient;

FIGS. 5A and 5B are side views illustrating the efferent contraction training device in FIG. 1 applied to an arm of a patient;

FIG. 6 is a graph showing a voltage waveform applied to an electrical stimulation pad of the efferent contraction training device in FIG. 1; and

FIG. 7 is a flow chart illustrating a training method using the training device in FIG. 1.

### DETAILED DESCRIPTION OF THE EMBODIMENTS

Various example embodiments will be described more fully hereinafter with reference to the accompanying drawings, in which some example embodiments are shown. The present invention may, however, be embodied in many different forms and should not be construed as limited to the example embodiments set forth herein. Rather, these example embodiments are provided so that this disclosure will be thorough and complete, and will fully convey the scope of the present invention to those skilled in the art. In the drawings, the sizes and relative sizes of layers and regions may be exaggerated for clarity.

It will be understood that when an element or layer is referred to as being “on,” “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numerals refer to like elements throughout. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular example embodiments only and is not intended to be limiting of the present invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Example embodiments are described herein with reference to cross-sectional illustrations that are schematic illustrations of idealized example embodiments (and intermediate structures). As such, variations from the shapes of the illustrations as a result, for example, of manufacturing techniques and/or tolerances, are to be expected. Thus, example embodiments should not be construed as limited to the particular shapes of regions illustrated herein but are to include deviations in shapes that result, for example, from manufacturing. For example, an implanted region illustrated as a rectangle will, typically, have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, example embodiments will be explained in detail with reference to the accompanying drawings.

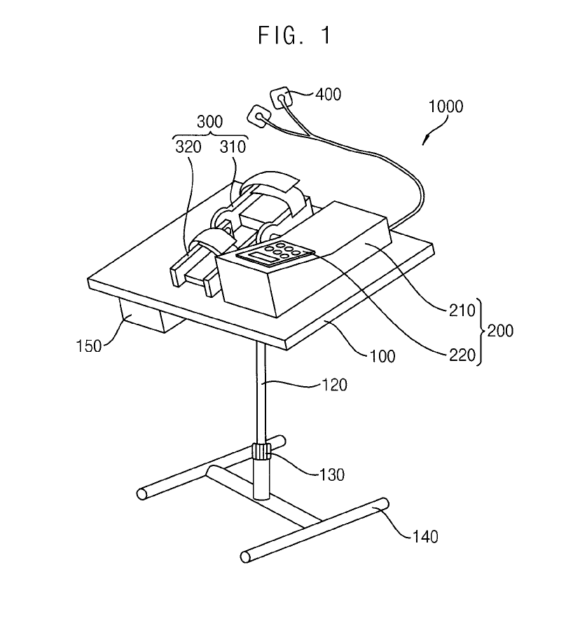

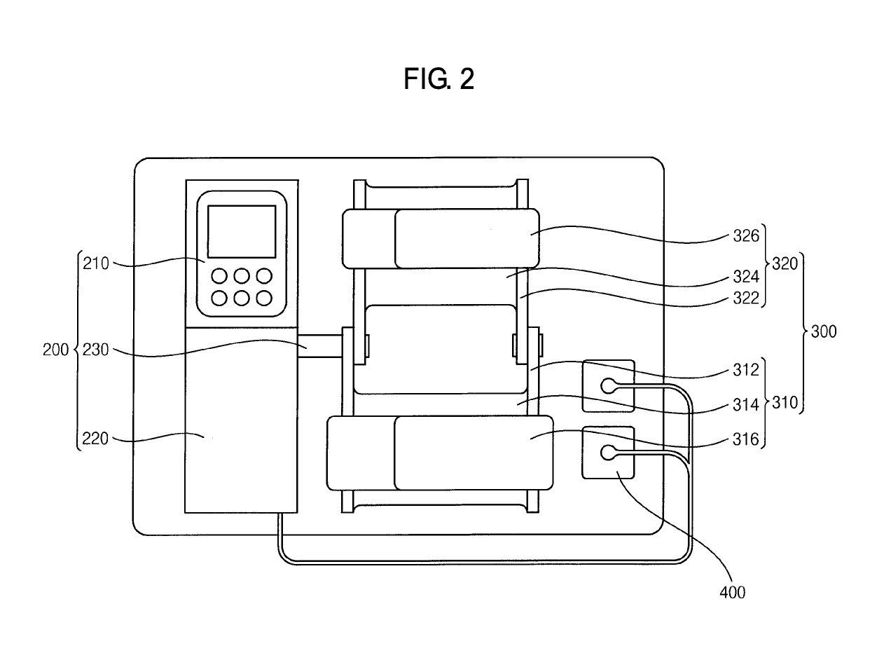

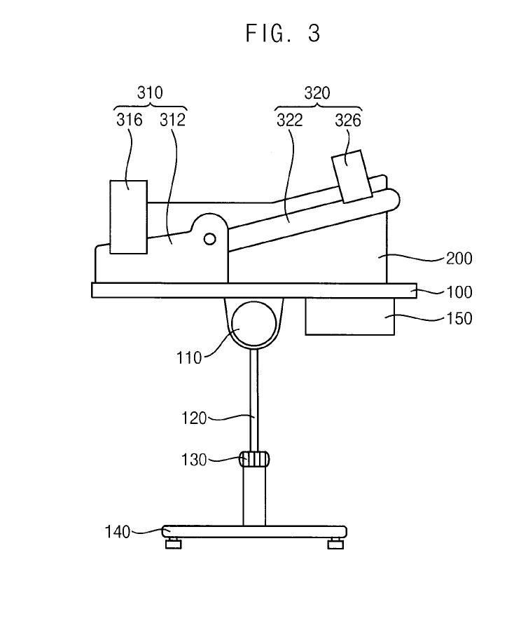

FIG. 1 is a perspective view illustrating an efferent contraction training device in accordance with example embodiments, FIG. 2 is a plan view illustrating the efferent contraction training device in FIG. 1, and FIG. 3 is a side view illustrating the efferent contraction training device in FIG. 1.

Referring to FIGS. 1 to 3, an efferent contraction training device 1000 of this example embodiment may include a table 100, a driving unit 200, a fixing unit 300 and an electrical stimulation pad 400.

The table 100 may be configured to support the driving unit 200 and the fixing unit 300. A first supporting rod 120 may be arranged under the table 100. A second supporting rod 140 may be arranged under the first supporting rod 120. A connecting rod 130 may be connected between the first supporting rod 120 and the second connecting rod 140. The connecting rod 130 may adjust a total length of the first connecting rod 120 and the second connecting rod 140. For example, the connecting rod 130 may be combined with the second connecting rod 140. The connecting rod 130 may fasten or unfasten the first supporting rod 120 to adjust a protruded length of the first supporting rod 120 from the second supporting rod 140.

An angle-adjusting unit 110 may be arranged between the table 100 and the first supporting rod 120. The angle-adjusting unit 110 may tilt the table 100 with respect to a horizontal plane. Further, the angle-adjusting unit 110 may fix the tilted table 100. Thus, the angle-adjusting unit 110 may provide the fixing unit 300 with a proper angle of a patient.

A sub-driving unit 150 may be arranged under the table 100. The sub-driving unit 150 may include an electrical circuit configured to drive the driving unit 200, the fixing unit 300 and/or the electrical stimulation pad 400. Alternatively, the electrical circuit may be installed in the driving unit 200. When the electrical circuit may not be mounted in the driving unit 200, the electrical circuit may be installed in the sub-driving unit 150.

The driving unit 200 may include an electrical circuit configured to drive the fixing unit 300 and the electrical stimulation pad 400. The driving unit 200 may apply a load to the fixing unit 300. The driving unit 200 may measure and display a torque generated from the fixing unit 300. The driving unit 200 may include a driving body 210 and a control display panel 220. The control display panel 220 may include control buttons for operating the efferent contraction training device 1000. The control display panel 220 may display information for training the patient. The driving unit 200 may further include a connecting member 230. The driving unit 200 may be connected with the fixing unit 300 via the connecting member 230. The driving unit 200 may transmit the torque to the fixing unit 300 through the connecting member 230. The driving unit 200 may apply a load to the rotating fixing unit 300 through the connecting member 230.

The fixing unit 200 may be arranged on the table 100. The fixing unit 200 may be configured to fix an articulation of the patient. The fixing unit 200 may include a first member 310 and a second member 320. The second member 320 may be connected with the first member 310 using a hinge. Thus, an angle may be formed between the first member 310 and the second member 320.

The first member 310 may include a first frame 312, a first cushion pad 314 and a first fixing band 316. The first cushion pad 314 may be configured to support an exercise portion of the patient. The first fixing band 316 may be configured to fix the exercise portion of the patient. The first fixing band 316 may include a Velcro type band.

The second member 320 may include a second frame 322, a second cushion pad 324 and a second fixing band 326. The second cushion pad 324 may be configured to support the exercise portion of the patient. The second fixing band 326 may be configured to fix the exercise portion of the patient. The second fixing band 326 may include a Velcro type band.

The first member 310 and the second member 320 may be configured to fix the articulation of the patient. The angle between the first member 310 and the second member 320 may be changed in accordance with movements of the articulation of the patient. The torque generated by the movement of the articulation may be transmitted to the driving unit 200 through the fixing unit 200 and the connecting member 230. The driving unit 200 may measure the torque. The driving unit 200 may apply the load to the rotating fixing unit 300 through the connecting member 230. Therefore, the proper load may be applied to the articulation of the patient.

The fixing member 300 may be configured to fix the articulation of the patient. For example, an arm of the patient may be fixed to the fixing member 300. An upper arm of the patient may be fixed to the first member 310. A lower arm of the patient may be fixed to the second member 320. When the electrical stimulation pad 400 may stimulate the patient, the arm of the patient may perform the efferent contraction exercise. The torque generated by changing the angle between the first member 310 and the second member 320 may be transmitted to the driving unit 200 through the connecting member 230. The driving unit 200 may measure the torque. The driving unit 200 may apply the load to the rotating fixing unit 300 through the connecting member 230 to set exercise strength suitable for the patient.

The electrical stimulation pad 400 may be electrically connected with the driving unit 200. The electrical stimulation pad 400 may closely make contact with the exercise portion of the patient. The electrical stimulation pad 400 may apply electrical stimulations to the exercise portion of the patient to induce the efferent contraction exercise. The efferent contraction exercise may be a slow uniform motion. The electrical stimulation pad 400 may apply the various electrical stimulations to the exercise portion of the patient by the driving unit 200.

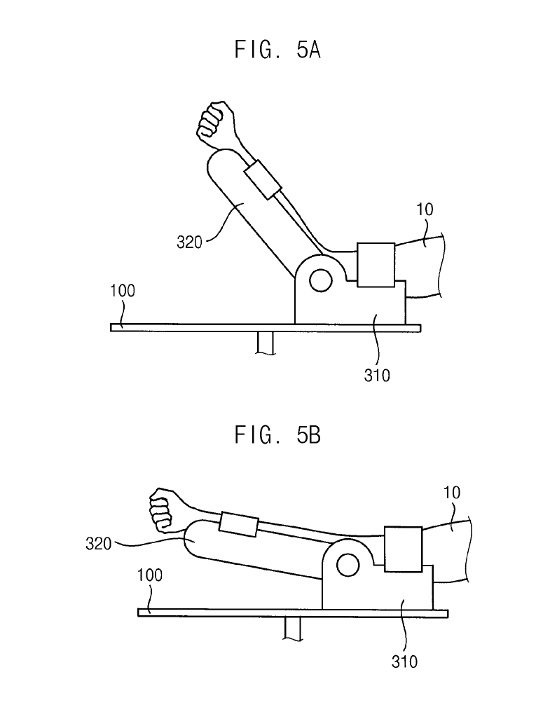

In example embodiments, the electrical stimulation may be a heteromorphic square wave having a frequency of about 20 Hz and a dwell time of about 200 μs between about +20V and about −20V. Referring to FIG. 6, the efferent contraction exercise may be the uniform motion having an angular speed of about 10°/s.

The efferent contraction exercise having the slow uniform motion may apply a muscular tension, which may be higher than a muscular tension of other exercises, to the exercise portion of the patient. Thus, the efferent contraction exercise may be effectively used for rehabilitating the patient with a paralytic musculature.

Moreover, the electrical stimulation pad of the efferent contraction training device may allow the articulation of the patient for the efferent contraction exercise having the slow uniform motion.

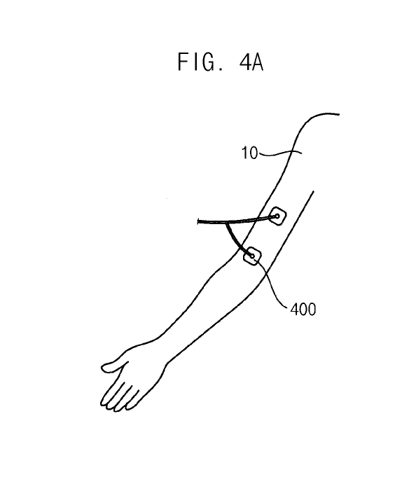

FIGS. 4A and 4B are perspective views illustrating the efferent contraction training device in FIG. 1 applied to an arm of a patient.

Referring to FIGS. 4A and 4B, the electrical stimulation pad 400 may be attached to the arm 10 of the patient. For example, in order to provide the arm 10 with the efferent contraction exercise, the electrical stimulation pad 400 may be attached to upper arm of the patient.

The angle of the table 100 may be adjusted in accordance with the body of the patient. The upper arm of the patient may be placed on the first cushion pad 314. The upper arm of the patient may be fixed using the first fixing band 316. The lower arm of the patient may be placed on the second cushion pad 324. The lower arm of the patient may be fixed using the second fixing band 326.

The electrical stimulation pad 400 may apply the electrical stimulation to the arm 10 of the patient by operating the driving unit 200 to provide the arm 10 with the efferent contraction exercise.

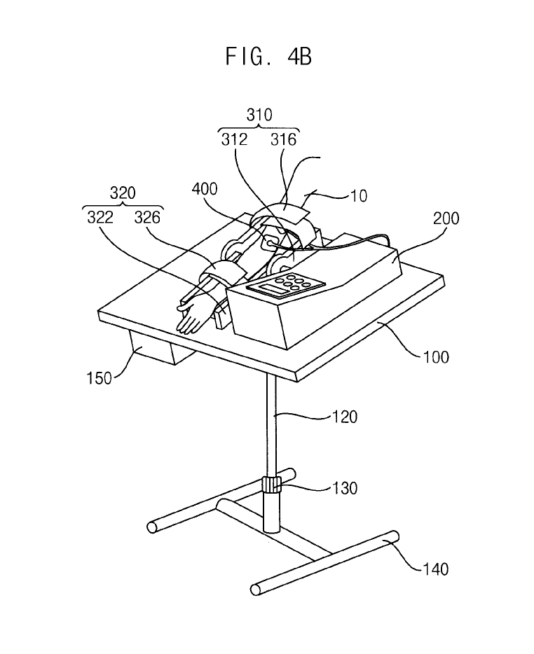

FIGS. 5A and 5B are side views illustrating the efferent contraction training device in FIG. 1 applied to an arm of a patient.

Referring to FIG. 5A, the arm 10 of the patient may be fixed to the first member 310 and the second member 320. The electrical stimulation pad 400 may apply the electrical stimulation to the folded arm 10 of the patient.

Referring to FIG. 5B, the arm 10 may perform the efferent contraction exercise by the electrical stimulation so that the angle between the first member 310 and the second member 320 may be increased to generate the torque. The torque may be transmitted to the driving unit 200 through the connecting member 230. The efferent contraction exercise of the arm 10 may be the slow uniform motion. For example, the efferent contraction exercise may be the uniform motion having an angular speed of about 10°/s. The driving unit 200 may apply the load to the rotating driving unit 300 through the connecting member 230 to control the exercise strength of the arm 10.

FIG. 6 is a graph showing a voltage waveform applied to an electrical stimulation pad of the efferent contraction training device in FIG. 1. In FIG. 6, an x-axis may represent a time of 100 μs by one gradation scale, and a y-axis may represent a voltage of 10V by one gradation scale.

Referring to FIG. 6, the voltage waveform applied to the electrical stimulation pad may be a heteromorphic square wave having a frequency of about 20 Hz and a dwell time of about 200 μs between about +20V and about −20V.

Thus, the exercise portion of the patient may perform the efferent contraction exercise having the slow uniform motion. For example, the electrical stimulation pad may apply the electrical stimulation to the arm of the patient to provide the arm with the efferent contraction exercise. The efferent contraction exercise of the arm 10 may be the slow uniform motion. For example, the efferent contraction exercise may be the uniform motion having an angular speed of about 10°/s. The voltage waveform applied to the electrical stimulation pad may be changed in accordance with the slow uniform motion.

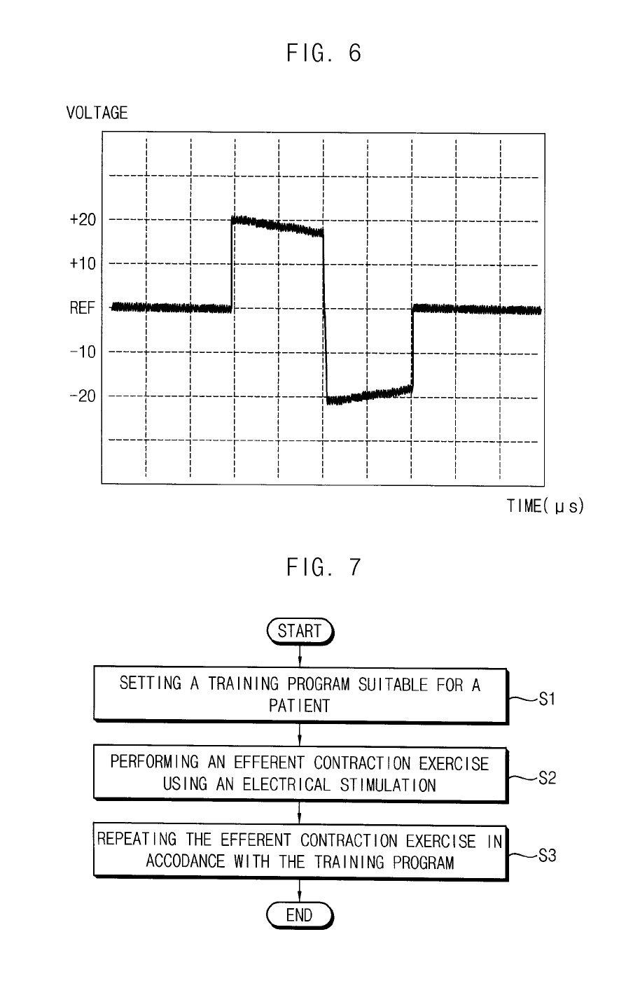

FIG. 7 is a flow chart illustrating a training method using the training device in FIG. 1.

Referring to FIG. 7, in step S1, a proper exercise program suitable for the patient may be set. In step S2, the efferent contraction training using the electrical stimulation may be performed. In step S3, the efferent contraction training may be repeated in accordance with the exercise program.

Setting the exercise program may include setting intensity of the electrical stimulation in accordance with the patient. For example, the intensity and the waveform of the electrical stimulation may be set to provide the arm of the patient with the efferent contraction exercise having the slow uniform motion. Setting the training program may include determining a voltage waveform of the electrical stimulation, voltage intensity, an exercise time, an exercise interval and an exercise period.

For example, the voltage waveform applied to the electrical stimulation pad may be a heteromorphic square wave having a frequency of about 20 Hz and a dwell time of about 200 μs between about +20V and about −20V.

Performing the efferent contraction exercise may include applying the electrical stimulation to the muscle of the articulation in the patient to generate the efferent contraction exercise. For example, the arm of the patient may perform the efferent contraction exercise having the slow uniform motion using the efferent contraction training device in FIG. 1.

Repeating the efferent contraction training may include continuing the efferent contraction training for the exercise time, and repeating the efferent contraction training the exercise numbers by the exercise interval. For example, the exercise time may be about 30 minutes. The exercise interval may be twice a week. The exercise period may be about 12 weeks.

According to example embodiments, the efferent contraction training device may provide the articulation of the patient with the efferent contraction exercise having a slow speed. The efferent contraction exercise having the slow speed may provide the articulation with a muscular tension stronger than a muscular tension generated by other exercises. Thus, the efferent contraction training device may be effectively used for rehabilitating the patient with the paralytic musculature.

Further, the efferent contraction training device may include the table having variable length and angle so that the efferent contraction training device may be adjusted to a body type of the patient.

Furthermore, the driving unit of the efferent contraction training device may measure the torques generated by the exercise of the patient and apply a proper load for setting an exercise intensity of the patient.

Moreover, the electrical stimulation pad of the efferent contraction training device may allow the articulation of the patient for the efferent contraction exercise having the slow uniform motion.

The foregoing is illustrative of example embodiments and is not to be construed as limiting thereof. Although a few example embodiments have been described, those skilled in the art will readily appreciate that many modifications are possible in the example embodiments without materially departing from the novel teachings and advantages of the present invention. Accordingly, all such modifications are intended to be included within the scope of the present invention as defined in the claims. In the claims, means-plus-function clauses are intended to cover the structures described herein as performing the recited function and not only structural equivalents but also equivalent structures. Therefore, it is to be understood that the foregoing is illustrative of various example embodiments and is not to be construed as limited to the specific example embodiments disclosed, and that modifications to the disclosed example embodiments, as well as other example embodiments, are intended to be included within the scope of the appended claims.

What is claimed is:

1. An efferent contraction training device comprising:
a fixing unit configured to fix an articulation of a patient, the fixing unit including a first member and a second member connected to the first member, and an angle between the first member and the second member being changed in accordance with an exercise of the articulation of the patient;
an electrical stimulation pad configured to apply an electrical stimulation to the articulation of the patient to provide the articulation with an efferent contraction exercise; and
a driving unit configured to drive the electrical stimulation pad.

2. The efferent contraction training device of claim 1, wherein the driving unit is configured to measure a torque generated when the angle is changed, and the driving unit comprises a control display panel configured to display the torque.

3. The efferent contraction training device of claim 1, wherein the driving unit is configured to apply a load to the fixing unit to rotate the first member and the second member.

4. The efferent contraction training device of claim 1,
wherein the first member comprises:
a first frame;
a first cushion pad configured to support the articulation of the patient; and
a first fixing band configured to fix the articulation of the patient on the first cushion pad, and
wherein the second member comprises:
a second frame;
a second cushion pad configured to support the articulation of the patient; and
a second fixing band configured to fix the articulation of the patient on the second cushion pad, and

[symbol]

5. The efferent contraction training device of claim 1, further comprising:
a table configured to support the fixing unit and the driving unit;
a first supporting rod arranged under the table;
a second supporting rod arranged under the first supporting rod; and
a connecting rod connected between the first supporting rod and the second supporting rod, the connecting rod configured to adjust a length of the first supporting rod and the second supporting rod.

6. The efferent contraction training device of claim 5, further comprising an angle-adjusting unit arranged between the table and the first supporting rod to adjust an angle between the table and the first supporting rod.

7. The efferent contraction training device of claim 1, wherein the efferent contraction exercise comprises a uniform motion.

8. The efferent contraction training device of claim 1, wherein the electrical stimulation pad applies a voltage to the articulation of the patient and a wave form of the voltage includes a heteromorphic square wave having a frequency of about 20 Hz and a dwell time of about 200 μus between about +20V and about −20V.

9. An efferent contraction training method comprising:
setting a training program suitable for a patient, the training program including a voltage waveform of an electrical stimulation applied to an articulation of a patient, a voltage intensity, an exercise time, an exercise interval and an exercise period;
applying the electrical stimulation of the articulation of the patient to provide the articulation with an efferent contraction exercise; and
repeating the efferent contraction exercise in accordance with the training program.

10. The efferent contraction training method of claim 9, wherein the efferent contraction exercise comprises a uniform motion having a speed of about 10°/s.

11. The efferent contraction training method of claim 9, wherein the electrical stimulation comprises a heteromorphic square wave having a frequency of about 20 Hz and a dwell time of about 200 μs between about +20V and about −20V.

12. The efferent contraction training method of claim 9, wherein the exercise time is be about 30 minutes, the exercise interval is twice a week, and the exercise period is about 12 weeks.
